(19) United States
(12) Patent Application Publication
(10) Pub. No.: US 2023/0381080 A1
(43) Pub. Date: Nov. 30, 2023

(54) USE OF PARTICULAR AMINO ACIDS AS A PRETREATMENT FOR A PROCESS FOR DYEING OR BLEACHING KERATIN FIBRES

(71) Applicant: L'Oreal, Paris (FR)

(72) Inventors: Thomas SENGMANY, SAINT-OUEN (FR); Chantal JOUY, SAINT-OUEN (FR); Leila HERCOUET, SAINT-OUEN (FR); Tristan VAUTIER, SAINT-OUEN (FR)

(21) Appl. No.: 18/265,749

(22) PCT Filed: Dec. 3, 2021
(86) PCT No.: PCT/EP2021/084188

§ 371 Date: Jun. 7, 2023

(30) Foreign Application Priority Data
Dec. 11, 2020 (FR) 2013092

(51) Int. Cl. A61K 8/44; A61K 8/34; A61K 8/81; A61K 8/898; A61Q 5/10; A61Q 5/08

(52) U.S. Cl. CPC A61K 8/44; A61K 8/34; A61K 8/8194; A61K 8/898; A61Q 5/10; A61Q 5/08

(57) ABSTRACT

The present invention relates to the use of one or more particular amino acids or of a composition comprising same as a pretreatment for a process for dyeing or bleaching keratin fibres in order to improve the dyeing or bleaching of the keratin fibres.

### FIELD OF THE INVENTION

The present invention relates to the use of one or more particular amino acids or of a composition comprising same as a pretreatment for a process for dyeing or bleaching keratin fibres in order to improve the dyeing or bleaching of the keratin fibres.

### BACKGROUND OF THE INVENTION

It is known practice to perform hair dyeing or bleaching processes in order to modify the colour of natural hair. These processes generally consist in applying to the keratin fibres hair compositions comprising chemical oxidizing agents and optionally direct dyes and/or oxidation dyes.

These dyeing or bleaching hair compositions admittedly have a dyeing or bleaching power, but the dyeing or bleaching performance levels may not always be satisfactory, notably in terms of colour build-up, intensity or uniformity or in terms of lightening, notably when the hair is sensitized by repeated chemical treatments such as dyeing operations, bleaching operations, permanent waves and/or relaxing operations and/or when the hair is laden with metals such as copper. Indeed, tap water can sometimes contain high contents of metals such as copper which, by accumulating on the hair over the course of successive washing operations, can have a negative impact on the results of a dyeing or bleaching operation.

It is therefore necessary to find a technical solution which makes it possible to overcome these various drawbacks, that is to say to improve the dyeing or bleaching of the hair, in particular hair sensitized by repeated chemical treatments and/or laden with metals such as copper.

The applicant has discovered, surprisingly, that all of these objectives can be achieved by means of the uses according to the present invention.

### SUMMARY OF THE INVENTION

According to a first aspect, a subject of the present invention is the use of one or more amino acids chosen from the compounds of formula (I₁), salts thereof and mixtures thereof:

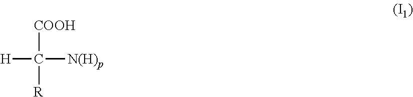

in which formula (I₁):
p is an integer equal to 1 or 2;
when p=1, R forms with the nitrogen atom a saturated 5- to 8-membered, preferably 5-membered, heterocycle, this ring possibly being optionally substituted with at least one group chosen from hydroxyl or (C₁-C₄)alkyl;
when p=2, R represents:
a hydrogen atom; or
a (C₁-C₁₂)alkyl group, preferably a (C₁-C₄)alkyl group, interrupted with at least one heteroatom or group chosen from —S—, —NH— or —C(NH)— and/or substituted with at least one group chosen from hydroxyl, amino or —NH—C(NH)—NH₂;
   as a pretreatment for a process for dyeing or bleaching keratin fibres in order to improve the dyeing or bleaching of the keratin fibres.

According to a second aspect, a subject of the present invention is the use of a composition (A) comprising i) one or more amino acids as defined above as a pretreatment for a process for dyeing or bleaching keratin fibres in order to improve the dyeing or bleaching of the keratin fibres.

According to a third aspect, a subject of the present invention is a process for improving the dyeing or bleaching of keratin fibres, comprising the application to the keratin fibres of one or more amino acids as defined above or of a composition (A) as defined above, as a pretreatment for a process for dyeing or bleaching keratin fibres.

### DETAILED DESCRIPTION OF THE INVENTION

For the purposes of the present invention, and unless otherwise indicated:
the term “keratin fibres” means fibres of human or animal origin, such as head hair, bodily hair, the eyelashes, the eyebrows, wool, angora, cashmere or fur. According to the present invention, the keratin fibres are preferably human keratin fibres, more preferentially the hair.
the term “sensitized keratin fibres” means keratin fibres embrittled by the action of chemical treatments such as dyeing operations, bleaching operations, permanent waves and/or relaxing operations and/or keratin fibres laden with metals such as copper, present for example at contents of at least 100 ppm.
the term “dyeing composition” means a composition comprising at least one colouring agent.
the term “bleaching composition” means a composition comprising at least one chemical oxidizing agent.
the term “alkyl group” means a linear or branched, saturated hydrocarbon-based radical.
the term “(Cₓ-C_{y})alkyl group” means an alkyl group comprising from x to y carbon atoms.
the term “colouring agent” means an oxidation dye, a direct dye or a pigment.
the term “oxidation dye” means an oxidation dye precursor chosen from oxidation bases and couplers. Oxidation bases and couplers are colourless or sparingly coloured compounds, which, via a condensation reaction in the presence of an oxidizing agent, give a coloured species.
the term “direct dye” means a natural and/or synthetic dye, including in the form of an extract or extracts, other than oxidation dyes. These are coloured compounds that will spread superficially on the fibre. They may be ionic or non-ionic, i.e. anionic, cationic, neutral or non-ionic.
the term “reducing agent” means an agent that is capable of reducing the disulfide bonds of the hair, such as compounds chosen from thiols, alkaline sulfites, hydrides and phosphines.
the term “chemical oxidizing agent” means an oxidizing agent other than atmospheric oxygen.

Unless otherwise indicated, when compounds are mentioned in the present patent application, this also includes the optical isomers thereof, the geometrical isomers thereof, the tautomers thereof, the salts thereof or the solvates thereof, alone or as a mixture.

The expressions “at least one” and “one or more” are synonymous and may be used interchangeably.

According to a first aspect, a subject of the present invention is the use of one or more amino acids chosen from the compounds of formula (I₁) as defined above, salts thereof and mixtures thereof, as a pretreatment for a process for dyeing keratin fibres in order to improve the dyeing of the keratin fibres or as a pretreatment for a process for bleaching keratin fibres in order to improve the bleaching of the keratin fibres.

According to a second aspect, a subject of the present invention is the use of a composition (A) comprising i) one or more amino acids chosen from the compounds of formula (I₁) as defined above, salts thereof and mixtures thereof, as a pretreatment for a process for dyeing keratin fibres in order to improve the dyeing of the keratin fibres or as a pretreatment for a process for bleaching keratin fibres in order to improve the bleaching of the keratin fibres.

The uses according to the present invention are non-therapeutic, cosmetic uses.

According to a third aspect, a subject of the present invention is a process for improving the dyeing or bleaching of keratin fibres, comprising the application to the keratin fibres of one or more amino acids chosen from the compounds of formula (I₁) as defined above, salts thereof and mixtures thereof, or of a composition (A) as defined above, as a pretreatment for a process for dyeing or bleaching keratin fibres.

According to one preferred embodiment, the amino acid(s) chosen from the compounds of formula (I₁) as defined above, salts thereof and mixtures thereof, or composition (A) as defined above are used as a pretreatment for a process for dyeing keratin fibres in order to improve the colour build-up and/or intensity and/or uniformity of the keratin fibres.

According to another preferred embodiment, the amino acid(s) chosen from the compounds of formula (I₁) as defined above, salts thereof and mixtures thereof, or composition (A) as defined above are used as a pretreatment for a process for bleaching keratin fibres in order to improve the lightening of the keratin fibres. Better neutralization of warm glints is in particular observed.

Preferably, the keratin fibres are sensitized keratin fibres.

According to one embodiment, the sensitized keratin fibres are keratin fibres embrittled by chemical dyeing and/or bleaching and/or permanent-waving and/or relaxing treatments.

According to another embodiment, the sensitized keratin fibres are keratin fibres laden with metals, preferably laden with metals at contents of at least 100 ppm.

According to one preferred embodiment, the keratin fibres are copper-laden keratin fibres, preferably laden with copper at contents of at least 100 ppm, more preferentially at contents of at least 200 ppm.

According to another embodiment, the keratin fibres are keratin fibres embrittled by chemical dyeing and/or bleaching and/or permanent-waving and/or relaxing treatments and laden with metals, preferably with copper.

### Amino Acids

The compounds of formula (I₁) may be in form of salts. These salts comprise the salts with organic or mineral bases, for example the salts of alkali metals, for instance the lithium, sodium or potassium salts; the salts of alkaline-earth metals, for instance the magnesium or calcium salts, and the zinc salts.

The compounds of formula (I₁) may be in the form of an optical isomer of L, D or DL configuration, preferably of L configuration.

As examples according to the present invention of compounds of formula (I₁) in the form of an optical isomer of L configuration, mention may be made of L-proline, L-methionine, L-serine, L-arginine and L-lysine.

Preferably, the amino acid(s) are chosen from glycine, proline, methionine, serine, arginine, lysine, salts thereof and mixtures thereof.

More preferentially, the amino acid(s) are chosen from glycine, proline, methionine, serine, salts thereof and mixtures thereof.

Even more preferentially, the amino acid is chosen from glycine, salts thereof and mixtures thereof.

As examples of glycine salts that may be used in the process according to the present invention, mention may be made of sodium glycinate, zinc glycinate, calcium glycinate, magnesium glycinate, manganese glycinate and potassium glycinate, preferably sodium glycinate or potassium glycinate.

According to one particularly preferred embodiment, the amino acid is glycine.

The amino acid(s) chosen from the compounds of formula (I₁), salts thereof and mixtures thereof are present in composition (A) in a total content of preferably at least 5% by weight, more preferentially of at least 8% by weight, relative to the total weight of composition (A).

The amino acid(s) chosen from the compounds of formula (I₁), salts thereof and mixtures thereof may be present in composition (A) in a total content ranging from 5% to 20% by weight, preferably ranging from 5% to 15% by weight, more preferentially ranging from 8% to 12% by weight, relative to the total weight of composition (A).

Composition (A) may preferably comprise at least 5% by weight, more preferentially at least 8% by weight, of glycine, salts thereof and mixtures thereof relative to the total weight of composition (A).

According to one preferred embodiment, composition (A) comprises from 5% to 20% by weight, preferably from 5% to 15% by weight, more preferentially from 8% to 12% by weight, of glycine, salts thereof and mixtures thereof relative to the total weight of composition (A).

Composition (A) may preferably comprise at least 5% by weight, more preferentially at least 8% by weight, of glycine, relative to the total weight of composition (A).

According to one preferred embodiment, composition (A) comprises from 5% to 20% by weight, preferably from 5% to 15% by weight, more preferentially from 8% to 12% by weight, of glycine relative to the total weight of composition (A).

pH

Composition (A) may have a pH ranging from 2 to 11. Preferably, the pH of composition (A) ranges from 4 to 10. More preferentially, the pH of composition (A) ranges from 8 to 10.

By way of example, the pH of composition (A) may be equal to 9.

The pH of composition (A) may be adjusted with at least one organic or mineral acid, or with at least one alkaline agent chosen from mineral or organic or hybrid alkaline agents and mixtures thereof.

The term “organic acid” means an acid, i.e. a compound that is capable of releasing a cation or proton H⁺ or H₃O⁺ in aqueous medium, which includes at least one optionally unsaturated, linear or branched C₁-C₂₀ hydrocarbon-based chain, a (hetero)cycloalkyl or (hetero)aryl group and at least one acidic chemical function chosen in particular from carboxyl C(O)OH, sulfonic SO₃H, sulfinic SO₂H, phosphonic PO₃H and phosphinic PO₂H₂.

More particularly, the organic or mineral acid used is chosen from hydrochloric acid HCl, hydrobromic acid HBr, sulfuric acid H₂SO₄, alkylsulfonic acids: (C₁-C₆)Alk-S(O)₂OH such as methylsulfonic acid and ethylsulfonic acid; arylsulfonic acids: Ar—S(O)₂OH such as benzenesulfonic acid and toluenesulfonic acid; (C₁-C₆)alkoxysulfinic acids: Alk-O—S(O)OH such as methoxysulfinic acid and ethoxysulfinic acid; aryloxysulfinic acids such as tolueneoxysulfinic acid and phenoxysulfinic acid; phosphoric acid H₃PO₄; triflic acid CF₃SO₃H; and tetrafluoroboric acid HBF₄, and the carboxylic acids of formula (II) below and the salts thereof:

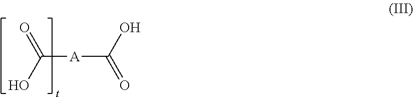

in which formula (II):

A represents a saturated or unsaturated, cyclic or noncyclic, aromatic or nonaromatic hydrocarbon-based group, which is monovalent when t is 0 or polyvalent when t is greater than or equal to 1, comprising from 1 to 50 carbon atoms, which is optionally interrupted with one or more heteroatoms and/or optionally substituted notably with one or more hydroxyl groups; preferably, A represents a monovalent (C₁-C₈)alkyl group or a polyvalent (C₁-C₆)alkylene group optionally substituted with one or more hydroxyl groups.

In particular, the acid used is chosen from the carboxylic acids of formula (II) as defined previously. Preferably, the acid used is an α-hydroxy acid such as lactic acid, glycolic acid, tartaric acid or citric acid.

The mineral alkaline agents are preferably chosen from aqueous ammonia, alkaline carbonates or bicarbonates such as sodium or potassium carbonates and sodium or potassium bicarbonates, sodium hydroxide or potassium hydroxide, and mixtures thereof.

The organic alkaline agents are preferably chosen from organic amines, i.e. they contain at least one substituted or unsubstituted amino group.

The organic alkaline agents are more preferentially chosen from organic amines with a pK_{b} at 25° C. of less than 12, preferably of less than 10, even more advantageously of less than 6. It should be noted that it is the pK_{b} corresponding to the function which has the highest basicity.

The organic alkaline agents are chosen, for example, from alkanolamines, oxyethylenated and/or oxypropylenated ethylenediamines and the compounds of formula (III) below:

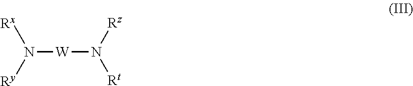

in which formula (III):
W is a divalent C₁-C₆ alkylene group optionally substituted with a hydroxyl group or a (C₁-C₆)alkyl group, and/or optionally interrupted with one or more heteroatoms such as oxygen or NR^{u};
R^{x}, R^{y}, R^{z}, R^{t} and R^{u}, which may be identical or different, represent a hydrogen atom or a group chosen from (C₁-C₆)alkyl, C₁-C₆ hydroxyalkyl or C₁-C₆ aminoalkyl.

Preferably, the alkanolamine is ethanolamine (or monoethanolamine).

In one variant of the invention, composition (A) comprises, as alkaline agent, one or more alkanolamines (preferably ethanolamine) and aqueous ammonia. In this variant, the alkanolamine(s) are present in a predominant amount relative to the aqueous ammonia.

Hybrid alkaline agents that may be mentioned include the salts of the amines mentioned previously with acids such as carbonic acid or hydrochloric acid.

### Cationic Polymers

Composition (A) may also comprise one or more cationic polymers.

The term “cationic polymer” means any polymer comprising cationic groups and/or groups that can be ionized into cationic groups, and not comprising any anionic groups and/or groups that can be ionized into anionic groups. Preferably, the cationic polymer is hydrophilic or amphiphilic.

The preferred cationic polymers are chosen from those that contain units including primary, secondary, tertiary and/or quaternary amine groups that may either form part of the main polymer chain or may be borne by a side substituent directly connected thereto.

The cationic polymers that may be used preferably have a weight-average molar mass (Mw) ranging from 500 to 5×10⁶ g/mol, preferably ranging from 103 to 3×10⁶ g/mol.

Preferably, composition (A) comprises one or more cationic polymers chosen from homopolymers or copolymers including in their structure one or more units corresponding to formula (I) or (II):

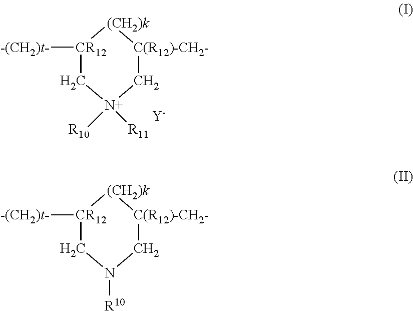

in which:
k and t are equal to 0 or 1, the sum k+t being equal to 1;
R₁₂ denotes a hydrogen atom or a methyl group;
R₁₀ and R₁₁, independently of each other, denote a (C₁-C₆)alkyl group, a C₁-C₅ hydroxyalkyl group, a C₁-C₄ amidoalkyl group; or alternatively R₁₀ and R₁₁ denote, together with the nitrogen atom to which they are attached, a heterocyclic group such as piperidyl or morpholinyl; preferably, R₁₀ and R₁₁, independently of each other, denote a (C₁-C₄)alkyl group;
Y⁻ is an anion, preferably chosen from bromide, chloride, acetate, borate, citrate, tartrate, bisulfate, bisulfite, sulfate and phosphate.

More preferentially, composition (A) comprises one or more cationic polymers chosen from homopolymers or copolymers including in their structure one or more units corresponding to formula (I) as defined previously.

Even more preferentially, composition (A) comprises one or more cationic polymers chosen from homopolymers of diallyldimethylammonium salts and copolymers of diallyldimethylammonium salts and of acrylamide.

Particularly preferably, composition (A) comprises one or more cationic polymers chosen from copolymers of diallyldimethylammonium salts and of acrylamide.

Mention may be made more particularly of the homopolymer of dimethyldiallylammonium salts (for example chloride) for example sold under the name Merquat 100 by the company Nalco, and the copolymers of diallyldimethylammonium salts (for example chloride) and of acrylamide, notably sold under the name Merquat 550 or Merquat 7SPR.

The cationic polymer(s) may be present in composition (A) in a total content ranging from 0.00001% to 5% by weight, preferably ranging from 0.00005% to 1% by weight, more preferentially ranging from 0.00007% to 0.5% by weight, relative to the total weight of composition (A).

### Amino Silicones

Composition (A) may also comprise one or more silicones, preferably chosen from amino silicones.

The term “amino silicone” means any silicone including at least one primary, secondary or tertiary amine function.

The weight-average molecular masses of these amino silicones may be measured by gel permeation chromatography (GPC) at ambient temperature (25° C.), as polystyrene equivalent. The columns used are μ styragel columns. The eluent is THF and the flow rate is 1 ml/min. 200 μl of a 0.5% by weight solution of silicone in THF are injected.

Detection is performed by refractometry and UV-metry.

Preferably, the amino silicones are chosen from the amino silicones of formula (B) below:

R′ₐG₃₋ₐ-Si(OSiG₂)ₙ-(OSiG_{b}R′_{2-b})ₘ—O—SiG₃₋ₐ-R′ₐ (B)

in which:
G, which may be identical or different, denotes a hydrogen atom or a group from among phenyl, OH, C₁-C₈ alkyl, for example methyl, or C₁-C₈ alkoxy, for example methoxy,
a, which may be identical or different, denotes 0 or an integer from 1 to 3, in particular 0,
b denotes 0 or 1, in particular 1,
m and n are numbers such that the sum (n+m) ranges from 1 to 2000, in particular from 50 to 150, n possibly denoting a number from 0 to 1999 and notably from 49 to 149, and m possibly denoting a number from 1 to 2000 and notably from 1 to 10,
R′, which may be identical or different, denotes a monovalent radical of formula —CqH2qL in which q is a number ranging from 2 to 8 and L is an optionally quaternized amine group chosen from the following groups:
N(R″)₂; —N*(R″)₃A-; —NR″-Q-N(R″)₂ and —NR″-Q-N—(R″)₃A-,
   in which R″, which may be identical or different, denotes hydrogen, phenyl, benzyl, or a saturated monovalent hydrocarbon-based radical, for example a C₁-C₂₀ alkyl radical; Q denotes a linear or branched group of formula CᵣH₂ᵣ, r being an integer ranging from 2 to 6, preferably from 2 to 4; and A⁻ represents a cosmetically acceptable anion, notably a halide such as fluoride, chloride, bromide or iodide.

More preferentially, the amino silicones are chosen from the amino silicones of formula (F) below:

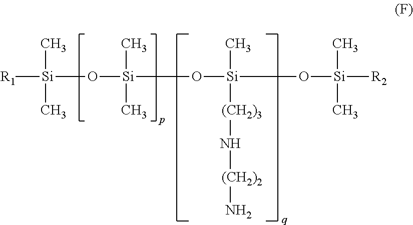

in which:
p and q are numbers such that the sum (p+q) ranges from 1 to 1000, in particular from 50 to 350, and more particularly from 150 to 250; p possibly denoting a number from 0 to 999 and notably from 49 to 349 and more particularly from 159 to 239, and q possibly denoting a number from 1 to 1000, notably from 1 to 10 and more particularly from 1 to 5;
R₁ and R₂, which are different, represent a hydroxyl or C₁-C₄ alkoxy radical, at least one of the radicals R₁ or R₂ denoting an alkoxy radical.

Preferably, the alkoxy radical is a methoxy radical.

The hydroxy/alkoxy mole ratio generally ranges from 1:0.8 to 1:1.1 and preferably from 1:0.9 to 1:1 and more particularly is equal to 1:0.95.

The weight-average molecular mass (Mw) of the silicone preferably ranges from 2000 to 200 000, even more particularly from 5000 to 100 000 and more particularly from 10 000 to 50 000.

The commercial products comprising silicones of structure (F) may include in their composition one or more other amino silicones of which the structure is different from formula (F).

A product containing amino silicones of structure (F) is sold by Wacker under the name Fluid WR 1300®.

Among the amino silicones of formula (F), mention may also be made of the product Belsil ADM Log 1 from Wacker.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or non-ionic. The number-average size of the silicone particles in the emulsion generally ranges from 3 nm to 500 nanometres.

The silicone(s) may be present in composition (A) in a total content ranging from 0.001% to 10% by weight, preferably ranging from 0.01% to 5% by weight, more preferentially ranging from 0.02% to 1% by weight, even more preferentially ranging from 0.05% to 0.5% by weight, relative to the total weight of composition (A).

The amino silicone(s) may be present in composition (A) in a total content ranging from 0.001% to 10% by weight, preferably ranging from 0.01% to 5% by weight, more preferentially ranging from 0.02% to 1% by weight, even more preferentially ranging from 0.05% to 0.5% by weight, relative to the total weight of composition (A).

Composition (A) preferably comprises a total content of colouring agents and/or reducing agents of less than 0.1% by weight, more preferentially of less than 0.01% by weight, even more preferentially of less than 0.001% by weight, relative to the total weight of composition (A).

According to a particularly preferred embodiment, composition (A) is free of colouring agents and/or reducing agents.

### Surfactants

Composition (A) preferably comprises less than 5% by weight, more preferentially less than 2% by weight, even more preferentially less than 1% by weight, of surfactants relative to the total weight of composition (A).

In particular, composition (A) may comprise a total content of anionic surfactants of less than 0.1% by weight, preferably of less than 0.01% by weight, more preferentially of less than 0.001% by weight, relative to the total weight of composition (A).

According to a particularly preferred embodiment, composition (A) is free of anionic surfactants.

Composition (A) may comprise a total content of non-ionic surfactants of less than 0.5% by weight relative to the total weight of composition (A).

### Monoalcohols

Preferably, composition (A) also comprises ii) one or more monoalcohols.

Composition (A) preferably comprises a total content of monoalcohols of at least 5% by weight, more preferentially of at least 8% by weight, relative to the total weight of composition (A).

The monoalcohols may be linear or branched.

The monoalcohol(s) are preferably chosen from C₂ to C₆ monoalcohols, more preferentially from C₂ to C₄ monoalcohols, even more preferentially from ethanol, isopropanol, tert-butanol, n-butanol and mixtures thereof.

According to a particularly preferred embodiment, the monoalcohol is ethanol.

The monoalcohol(s) may be present in composition (A) in a total content ranging from 5% to 20% by weight, preferably ranging from 5% to 15% by weight, more preferentially ranging from 8% to 12% by weight, relative to the total weight of composition (A).

### Organic Solvents

Composition (A) may comprise, in addition to the monoalcohol(s), at least one organic solvent chosen from polyols, polyol ethers and mixtures thereof.

The polyols are preferably chosen from propylene glycol, dipropylene glycol, glycerol and mixtures thereof.

The polyol ethers are preferably chosen from propylene glycol monomethyl ether, diethylene glycol monomethyl ether and monoethyl ether and mixtures thereof.

Composition (A) may comprise a total content of organic solvents other than monoalcohols ranging from 1% to 40% by weight, preferably ranging from 5% to 30% by weight, more preferentially ranging from 8% to 15% by weight, relative to the total weight of composition (A).

### Water

Composition (A) may comprise a total content of water ranging from 1% to 90% by weight, preferably ranging from 20% to 90% by weight, more preferentially ranging from 40% to 90% by weight, even more preferentially ranging from 60% to 85% by weight, relative to the total weight of composition (A).

### Dyeing or Bleaching Process

The dyeing process comprises the application of a dyeing composition to the keratin fibres. The bleaching process comprises the application of a bleaching composition to the keratin fibres.

### Dyeing or Bleaching Composition

### Chemical Oxidizing Agent

The dyeing or bleaching composition applied to the keratin fibres may comprise at least one chemical oxidizing agent.

Preferably, the chemical oxidizing agent is chosen from hydrogen peroxide, urea peroxide, alkali metal bromates, peroxygenated salts, peracids and precursors thereof, and mixtures thereof.

More preferentially, the chemical oxidizing agent is chosen from hydrogen peroxide, peroxygenated salts, and mixtures thereof.

Even more preferentially, the chemical oxidizing agent is chosen from hydrogen peroxide, persulfates, perborates, percarbonates of alkali metals or alkaline-earth metals or of ammonium, and mixtures thereof.

A chemical oxidizing agent that is particularly preferred is hydrogen peroxide.

Examples of peroxygenated salts that may be mentioned include sodium, potassium or ammonium persulfates and mixtures thereof.

When the composition is a bleaching composition, it may preferably comprise hydrogen peroxide and a peroxygenated salt.

The dyeing or bleaching composition may comprise a total content of chemical oxidizing agents ranging from 0.5% to 60% by weight, preferably ranging from 0.5% to 40% by weight, more preferentially ranging from 1% to 30% by weight, relative to the total weight of the dyeing or bleaching composition.

### Liquid Fatty Substances

The dyeing or bleaching composition may also comprise one or more fatty substances that are liquid at ambient temperature (25° C.) and at atmospheric pressure (1.013×10⁵ Pa), other than salified fatty acids.

The term “fatty substance” means an organic compound that is insoluble in water at ambient temperature (25° C.) and at atmospheric pressure (1.013×10⁵ Pa) (solubility of less than 5% by weight, preferably of less than 1% by weight and even more preferentially of less than 0.1% by weight). They bear in their structure at least one hydrocarbon-based chain including at least 6 carbon atoms and/or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

The term “oil” means a “fatty substance” that is liquid at ambient temperature (25° C.) and at atmospheric pressure (1.013×10⁵ Pa).

The term “nonsilicone fatty substance” refers to a fatty substance not containing any Si—O bonds and the term “silicone fatty substance” refers to a fatty substance containing at least one Si—O bond.

The liquid fatty substances that may be used in the dyeing or bleaching composition are different from salified fatty acids, i.e. they can be present in the composition in the form of free fatty acids. In other words, these fatty substances do not contain any salified carboxylic acid groups (—C(O)O—). In particular, these fatty substances are neither polyoxyalkylenated nor polyglycerolated.

Preferably, the fatty substances are different from non-salified fatty acids.

More particularly, the liquid fatty substances according to the invention are chosen from C₆ to C₁₆ liquid hydrocarbons, liquid hydrocarbons comprising more than 16 carbon atoms, nonsilicone oils of animal origin, oils of triglyceride type of plant or synthetic origin, fluoro oils, liquid fatty alcohols, liquid fatty acid and/or fatty alcohol esters other than triglycerides, and silicone oils, and mixtures thereof.

It is recalled that the fatty alcohols and esters more particularly contain at least one saturated or unsaturated, linear or branched hydrocarbon-based group, comprising 6 to 30 and better still from 8 to 30 carbon atoms, which is optionally substituted, in particular with one or more hydroxyl groups (in particular 1 to 4). If they are unsaturated, these compounds may comprise one to three conjugated or unconjugated carbon-carbon double bonds.

As regards the C₆ to C₁₆ liquid hydrocarbons, they are linear, branched or optionally cyclic, and are preferably alkanes. Examples that may be mentioned include hexane, cyclohexane, undecane, dodecane, isododecane, tridecane or isoparaffins, such as isohexadecane or isodecane, and mixtures thereof.

The liquid hydrocarbons comprising more than 16 carbon atoms may be linear or branched, and of mineral or synthetic origin, and are preferably chosen from liquid paraffin or liquid petroleum jelly, polydecenes, hydrogenated polyisobutene such as Parleam®, and mixtures thereof.

A hydrocarbon-based oil of animal origin that may be mentioned is perhydrosqualene.

The triglyceride oils of plant or synthetic origin are preferably chosen from liquid fatty acid triglycerides including from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, sunflower oil, castor oil, avocado oil, caprylic/capric acid triglycerides, for instance those sold by the company Stéarinerie Dubois or those sold under the names Miglyol® 810, 812 and 818 by the company Dynamit Nobel, jojoba oil and shea butter oil, and mixtures thereof.

As regards the fluoro oils, they may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or else bromoperfluorooctyl sold under the name

Foralkyl® by the company Atochem; nonafluoromethoxybutane and nonafluoroethoxyisobutane; perfluoromorpholine derivatives, such as 4-trifluoromethylperfluoromorpholine sold under the name PF 5052® by the company 3M. The liquid fatty alcohols may more particularly be chosen from linear or branched, saturated or unsaturated alcohols, preferably unsaturated or branched alcohols, including from 6 to 30 carbon atoms, preferably from 8 to 30 carbon atoms. Examples that may be mentioned include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol and linoleyl alcohol, and mixtures thereof.

As regards the liquid esters of fatty acids and/or of fatty alcohols, other than the triglycerides mentioned above, mention may be made notably of the esters of saturated or unsaturated, linear C₁ to C₂₆ or branched C₃ to C₂₆ aliphatic monoacids or polyacids and of saturated or unsaturated, linear C₁ to C₂₆ or branched C₃ to C₂₆ aliphatic monoalcohols or polyalcohols, the total carbon number of the esters being greater than or equal to 6 and more advantageously greater than or equal to 10.

Preferably, for the esters of monoalcohols, at least one from among the alcohol and the acid from which the esters of the invention are derived is branched.

Among the monoesters, mention may be made of dihydroabietyl behenate; octyldodecyl behenate;

isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononanoate; octyldodecyl erucate; oleyl erucate; ethyl palmitate, isopropyl palmitate, 2-ethylhexyl palmitate, 2-octyldecyl palmitate, alkyl myristates such as isopropyl 2-octyldodecyl myristate, isobutyl stearate; 2-hexyldecyl laurate, and mixtures thereof.

Preferably, among the monoesters of monoacids and of monoalcohols, use will be made of ethyl palmitate, isopropyl palmitate, alkyl myristates such as isopropyl myristate or ethyl myristate, isocetyl stearate, 2-ethylhexyl isononanoate, isodecyl neopentanoate, and isostearyl neopentanoate, and mixtures thereof.

Still within the context of this variant, use may also be made of esters of C₄ to C₂₂ dicarboxylic or tricarboxylic acids and of C₁ to C₂₂ alcohols and esters of mono-, di- or tricarboxylic acids and of C₂ to C₂₆ di-, tri-, tetra- or pentahydroxy alcohols.

Mention may notably be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates, and mixtures thereof.

The dyeing or bleaching composition may also comprise, as fatty ester, sugar esters and diesters of C₆ to C₃₀ and preferably C₁₂ to C₂₂ fatty acids. It is recalled that the term “sugar” refers to oxygen-bearing hydrocarbon-based compounds bearing several alcohol functions, with or without aldehyde or ketone functions, and which include at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen notably from the group comprising the esters or mixtures of esters of sugars described previously and of linear or branched, saturated or unsaturated C₆ to C₃₀ and preferably C₁₂ to C₂₂ fatty acids. If they are unsaturated, these compounds may comprise one to three conjugated or unconjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates, arachidonates or mixtures thereof, for instance especially the mixed oleo-palmitate, oleo-stearate and palmito-stearate esters.

More particularly, use is made of monoesters and diesters and especially sucrose, glucose or methylglucose mono- or di-oleates, stearates, behenates, oleopalmitates, linoleates, linolenates and oleostearates, and mixtures thereof.

An example that may be mentioned is the product sold under the name Glucate® DO by the company Amerchol, which is a methylglucose dioleate.

Preferably, use will be made of a liquid ester of a monoacid and of a monoalcohol.

The silicone oils that may be used in the dyeing or bleaching composition may be volatile or non-volatile, cyclic, linear or branched silicone oils, which are unmodified or modified with organic groups, and preferably have a viscosity from 5×10⁻⁶ to 2.5 m²/s at 25° C., and preferably 1×10⁻⁵ to 1 m²/s.

Preferably, the silicone oils are chosen from polydialkylsiloxanes, especially polydimethylsiloxanes (PDMS), and liquid polyorganosiloxanes including at least one aryl group.

These silicone oils may also be organomodified. The organomodified silicone oil(s) that may be used in the dyeing or bleaching composition are preferably liquid silicones as defined previously and including in their structure one or more organofunctional groups attached via a hydrocarbon-based group, chosen, for example, from amine groups and alkoxy groups.

Organopolysiloxanes are defined in greater detail in Walter NOLL's “Chemistry and Technology of Silicones” (1968), Academic Press. They may be volatile or non-volatile.

When they are volatile, the silicone oils are more particularly chosen from those with a boiling point of between 60° C. and 260° C., and even more particularly from:

(i) cyclic polydialkylsiloxanes including from 3 to 7 and preferably from 4 to 5 silicon atoms. These are, for example, octamethylcyclotetrasiloxane sold especially under the name VOLATILE SILICONE® 7207 by UNION CARBIDE or SILBIONE® 70045 V2 by RHODIA, decamethylcyclopentasiloxane sold under the name VOLATILE SILICONE® 7158 by UNION CARBIDE, and SILBIONE® 70045 V5 by RHODIA, and mixtures thereof.

Mention may also be made of cyclocopolymers of the dimethylsiloxane/methylalkylsiloxane type, such as VOLATILE SILICONE® FZ 3109 sold by the company UNION CARBIDE.

Mention may also be made of mixtures of cyclic polydialkylsiloxanes with organosilicon compounds, such as the mixture of octamethylcyclotetrasiloxane and tetra(trimethylsilyl)pentaerythritol (50/50) and the mixture of octamethylcyclotetrasiloxane and oxy-1,1-bis(2,2,2′,2′,3,3′-hexatrimethylsilyloxy)neopentane;

(ii) linear volatile polydialkylsiloxanes containing 2 to 9 silicon atoms and having a viscosity of less than or equal to 5×10⁻⁶ m²/s at 25° C. An example is decamethyltetrasiloxane sold especially under the name “SH 200” by the company TORAY SILICONE. Silicones falling within this category are also described in the article published in Cosmetics and Toiletries, Vol. 91, January 76, pp. 27-32, TODD & BYERS, “Volatile Silicone Fluids for Cosmetics”.

Non-volatile polydialkylsiloxanes are preferably used.

These silicone oils are more particularly chosen from polydialkylsiloxanes, among which mention may be made mainly of polydimethylsiloxanes bearing trimethylsilyl end groups.

The viscosity of the silicones is measured at 25° C. according to the standard ASTM 445, Appendix C.

Among these polydialkylsiloxanes, mention may be made, in a nonlimiting manner, of the following commercial products:
the SILBIONE® oils of the 47 and 70 047 series or the MIRASIL® oils sold by RHODIA, such as, for example, the oil 70 047 V 500 000;
the oils of the MIRASIL® series sold by the company RHODIA;
the oils of the 200 series from the company DOW CORNING, such as DC200 with a viscosity of 60 000 mm²/s;
the VISCASIL® oils from GENERAL ELECTRIC and certain oils of the SF series (SF 96, SF 18) from GENERAL ELECTRIC.

Mention may also be made of polydimethylsiloxanes bearing dimethylsilanol end groups, known under the name dimethiconol (CTFA), such as the oils of the 48 series from the company RHODIA.

The organomodified silicones that may be used in the dyeing or bleaching composition are silicones as defined previously and including in their structure one or more organofunctional groups attached via a hydrocarbon-based group.

As regards the liquid polyorganosiloxanes including at least one aryl group, they may especially be polydiphenylsiloxanes, and polyalkylarylsiloxanes functionalized with the organofunctional groups mentioned previously.

The polyalkylarylsiloxanes are particularly chosen from linear and/or branched polydimethyl/methylphenylsiloxanes and polydimethyl/diphenylsiloxanes with a viscosity ranging from 1×10⁻⁵ to 5×10⁻² m²/s at 25° C.

Among these polyalkylarylsiloxanes, examples that may be mentioned include the products sold under the following names:
the SILBIONE® oils of the 70 641 series from RHODIA;
the oils of the RHODORSIL® 70 633 and 763 series from RHODIA;
the oil DOW CORNING 556 COSMETIC GRADE FLUID from DOW CORNING;
the silicones of the PK series from BAYER, such as the product PK20;
the silicones of the PN and PH series from BAYER, such as the products PN1000 and PH1000;
certain oils of the SF series from GENERAL ELECTRIC, such as SF 1023, SF 1154, SF 1250 and SF 1265.

Among the organomodified silicones, mention may be made of polyorganosiloxanes including:
substituted or unsubstituted amine groups, such as the products sold under the names GP 4 Silicone Fluid and GP 7100 by the company GENESEE or the products sold under the names Q2 8220 and DOW CORNING 929 or 939 by the company DOW CORNING.

The substituted amine groups are, in particular, C₁ to C₄ aminoalkyl groups;
alkoxy groups;
hydroxyl groups.

The liquid fatty substance(s) are preferentially chosen from liquid hydrocarbons containing more than 16 carbon atoms, plant oils, liquid fatty alcohols and liquid fatty esters, silicone oils and mixtures thereof.

Preferentially, the liquid fatty substance(s) are chosen from liquid hydrocarbons comprising more than 16 carbon atoms, in particular liquid petroleum jelly.

In a particular embodiment, the total content of liquid fatty substances included in the dyeing or bleaching composition is greater than or equal to 20% by weight, preferably greater than or equal to 30% by weight, more preferentially greater than or equal to 35% by weight, relative to the total weight of the dyeing or bleaching composition.

More preferentially, the total content of liquid fatty substances included in the dyeing or bleaching composition ranges from 20% to 80% by weight, and preferably from 30% to 70% by weight, relative to the total weight of the dyeing or bleaching composition.

### Alkaline Agents

The dyeing or bleaching composition may optionally also comprise one or more alkaline agents.

Preferably, the dyeing or bleaching composition comprises one or more organic or mineral alkaline agents.

The mineral alkaline agent(s) are preferably chosen from aqueous ammonia, alkali metal carbonates or bicarbonates such as sodium or potassium carbonates and sodium or potassium bicarbonates, sodium or potassium hydroxide, alkali metal silicates or metasilicates such as sodium or potassium silicates or metasilicates, or mixtures thereof.

The organic alkaline agent(s) are preferably chosen from organic amines with a pK_{b} at 25° C. of less than 12, preferably of less than 10, and even more advantageously of less than 6. It should be noted that it is the pK_{b} corresponding to the function which has the highest basicity. In addition, the organic amines do not comprise any alkyl or alkenyl fatty chains comprising more than ten carbon atoms.

The organic alkaline agent(s) are chosen, for example, from alkanolamines, oxyethylenated and/or oxypropylenated ethylenediamines, amino acids and the compounds of formula (III) below:

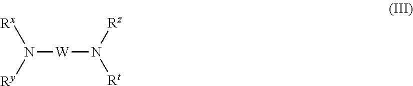

in which formula (III):
W is a divalent C₁-C₆ alkylene group optionally substituted with a hydroxyl group or a (C₁-C₆)alkyl group, and/or optionally interrupted with one or more heteroatoms such as oxygen or NR^{u};
R^{x}, R^{y}, R^{z}, R^{t} and R^{u}, which may be identical or different, represent a hydrogen atom or a group chosen from (C₁-C₆)alkyl, C₁-C₆ hydroxyalkyl or C₁-C₆ aminoalkyl.

Examples of amines of formula (III) that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁ to C₈ alkyl groups bearing one or more hydroxyl radicals.

Organic amines chosen from alkanolamines such as monoalkanolamines, dialkanolamines or trialkanolamines comprising one to three identical or different C₁ to C₄ hydroxyalkyl radicals are in particular suitable for performing the invention.

Among the compounds of this type, mention may be made of monoethanolamine (MEA), diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethylamino)methane.

More particularly, the amino acids that may be used are of natural or synthetic origin, in their L, D or racemic form, and include at least one acid function chosen more particularly from carboxylic acid, sulfonic acid, phosphonic acid and phosphoric acid functions. The amino acids may be in neutral or ionic form.

As amino acids that may be used in the dyeing or bleaching composition, mention may notably be made of aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine and valine.

Advantageously, the amino acids are basic amino acids comprising an additional amine function optionally included in a ring or in a ureido function.

Such basic amino acids are preferably chosen from those corresponding to formula (IV) below, and also salts thereof:

R—CH₂—CH(NH₂)—C(O)—OH (IV)

in which R represents a group chosen from imidazolyl, preferably imidazolyl-4-yl; aminopropyl; aminoethyl; —(CH₂)₂N(H)—C(O)—NH₂; and —(CH₂)₂—N(H)—C(NH)—NH₂.

The compounds corresponding to formula (IV) are histidine, lysine, arginine, ornithine and citrulline.

The organic amine may also be chosen from organic amines of heterocyclic type. Besides histidine that has already been mentioned in the amino acids, mention may in particular be made of pyridine, piperidine, imidazole, triazole, tetrazole and benzimidazole.

The organic amine may also be chosen from amino acid dipeptides. As amino acid dipeptides that may be used in the present invention, mention may notably be made of carnosine, anserine and balenine.

The organic amine may also be chosen from compounds including a guanidine function. As amines of this type that may be used in the present invention, besides arginine which has already been mentioned as an amino acid, mention may be made notably of creatine, creatinine, 1,1-dimethylguanidine, 1,1-diethylguanidine, glycocyamine, metformin, agmatine, n-amidinoalanine, 3-guanidinopropionic acid, 4-guanidinobutyric acid and 2-([amino(imino)methyl]amino)ethane-1-sulfonic acid.

Hybrid compounds that may be mentioned include the salts of the amines mentioned previously with acids such as carbonic acid or hydrochloric acid.

Guanidine carbonate or monoethanolamine hydrochloride may be used in particular. Preferably, the alkaline agent(s) present in the dyeing or bleaching composition are chosen from aqueous ammonia, alkanolamines, alkali metal silicates, alkali metal metasilicates and mixtures thereof.

More preferentially, the alkaline agent present in the dyeing composition is monoethanolamine.

More preferentially, the alkaline agent present in the bleaching composition is chosen from sodium silicate, sodium metasilicate and mixtures thereof.

The total content of alkaline agents included in the dyeing or bleaching composition may range from 0.01% to 30% by weight, preferably from 0.1% to 20% by weight, relative to the total weight of the dyeing or bleaching composition.

### Solvents

The dyeing or bleaching composition may optionally also comprise one or more organic solvents.

Examples of organic solvents that may be mentioned include linear or branched C₂ to C₄ alkanols, such as ethanol and isopropanol; glycerol; polyols and polyol ethers, for instance 2-butoxyethanol, propylene glycol, hexylene glycol, dipropylene glycol, propylene glycol monomethyl ether, diethylene glycol monomethyl ether and monoethyl ether, and also aromatic alcohols or ethers, such as benzyl alcohol or phenoxyethanol, and mixtures thereof.

The organic solvent(s) may be present in the dyeing or bleaching composition in a content ranging from 0.01% to 30% by weight, preferably ranging from 2% to 25% by weight, relative to the total weight of the dyeing or bleaching composition.

### Dyeing Composition

The dyeing composition may comprise at least one colouring agent chosen from oxidation dyes, direct dyes, and mixtures thereof, preferably from oxidation dyes.

### Oxidation Dyes

The oxidation dyes are generally chosen from one or more oxidation bases, optionally combined with one or more coupling agents (also known as couplers).

### Oxidation Bases

The dyeing composition may optionally comprise one or more oxidation bases advantageously chosen from those conventionally used in the dyeing of keratin fibres.

By way of example, the oxidation bases are chosen from para-phenylenediamines, bis(phenyl)alkylenediamines, para-aminophenols, ortho-aminophenols and heterocyclic bases, and the corresponding addition salts.

Among the para-phenylenediamines that may be mentioned are, for example, para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the corresponding addition salts with an acid.

Among the para-phenylenediamines mentioned above, para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the corresponding addition salts with an acid, are particularly preferred.

Among the bis(phenyl)alkylenediamines that may be mentioned are, for example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the corresponding addition salts.

Among the para-aminophenols that are mentioned are, for example, para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(p-hydroxyethylaminomethyl)phenol and 4-amino-2-fluorophenol, and the corresponding addition salts with an acid.

Among the ortho-aminophenols that may be mentioned are, for example, 2-aminophenol, 2-amino-5-methylphenol, 2-amino-6-methylphenol and 5-acetamido-2-aminophenol, and the corresponding addition salts.

Among the heterocyclic bases that may be mentioned are, for example, pyridine, pyrimidine and pyrazole derivatives.

Among the pyridine derivatives that may be mentioned are the compounds described, for example, in patents GB 1 026 978 and GB 1 153 196, for example 2,5-diaminopyridine, 2-(4-methoxyphenyl)amino-3-aminopyridine and 3,4-diaminopyridine, and the corresponding addition salts.

Other pyridine oxidation bases that are useful in the present invention are the 3-aminopyrazolo[1,5-a]pyridine oxidation bases or the corresponding addition salts described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-β-hydroxyethoxy-3-aminopyrazolo[1,5-a]pyridine and 2-(4-dimethylpiperazinium-1-yl)-3-aminopyrazolo[1,5-a]pyridine, and the corresponding addition salts.

More particularly, the oxidation bases that are useful in the present invention are chosen from 3-aminopyrazolo[1,5-a]pyridines and are preferably substituted on carbon atom 2 with:
a) a (di)(C₁-C₆)(alkyl)amino group, said alkyl group possibly being substituted with at least one hydroxyl, amino or imidazolium group;
b) an optionally cationic 5- to 7-membered heterocycloalkyl group comprising from 1 to 3 heteroatoms, optionally substituted with one or more (C₁-C₆)alkyl groups, such as a di(C₁-C₄)alkylpiperazinium group; or
c) a (C₁-C₆)alkoxy group optionally substituted with one or more hydroxyl groups, such as a β-hydroxyalkoxy group, and the corresponding addition salts.

Among the pyrimidine derivatives that may be mentioned are the compounds described, for example, in patents DE 2359399; JP 88-169571; JP 05-63124; EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof and the tautomeric forms thereof, when a tautomeric equilibrium exists.

Among the pyrazole derivatives that may be mentioned are the compounds described in patents DE 3843892 and DE 4133957 and patent applications WO 94/08969, WO 94/08970, FR-A-2 733 749 and DE 195 43 988, for instance 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the corresponding addition salts. Use may also be made of 4,5-diamino-1-(β-methoxyethyl)pyrazole.

A 4,5-diaminopyrazole will preferably be used and even more preferentially 4,5-diamino-1-(β-hydroxyethyl)pyrazole and/or a corresponding salt.

The pyrazole derivatives that may also be mentioned comprise diamino-N,N-dihydropyrazolopyrazolones and in particular those described in patent application FR-A-2 886 136, such as the following compounds and the corresponding addition salts: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-di-(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one and 2,3-diamino-6-hydroxy-6,7-dihydro-1H,5H-pyrazolo[1,2-a]pyrazol-1-one.

Use will preferably be made of 2,3-diamino-6,7-dihydro-1H,5H-pyrazolo[1,2-a]pyrazol-1-one and/or a corresponding salt.

Heterocyclic bases that will preferably be used are 4,5-diamino-1-(β-hydroxyethyl)pyrazole and/or 2,3-diamino-6,7-dihydro-1H,5H-pyrazolo[1,2-a]pyrazol-1-one and/or a corresponding salt.

### Coupling Agents

The dyeing composition may optionally comprise one or more coupling agents advantageously chosen from those conventionally used in the dyeing of keratin fibres. Among these coupling agents, mention may be made in particular of meta-phenylenediamines, meta-aminophenols, meta-diphenols, naphthalene-based coupling agents and heterocyclic coupling agents, and also the corresponding addition salts. Mention may be made, for example, of 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(3-hydroxyethylamino)toluene, 6-hydroxyindoline, 2,6-dihydroxy-4-methylpyridine, 1-H-3-methylpyrazol-5-one, 1-phenyl-3-methylpyrazol-5-one, 2,6-dimethylpyrazolo[1,5-b]-1,2,4-triazole, 2,6-dimethyl[3,2-c]-1,2,4-triazole and 6-methylpyrazolo[1,5-a]benzimidazole, 2-methyl-5-aminophenol, 5-N-(β-hydroxyethyl)amino-2-methylphenol, 3-aminophenol and 3-amino-2-chloro-6-methylphenol, the corresponding addition salts with an acid and the corresponding mixtures.

In general, the addition salts of oxidation bases and coupling agents that may be used in the context of the invention are chosen in particular from the addition salts with an acid, such as the hydrochlorides, hydrobromides, sulfates, citrates, succinates, tartrates, lactates, tosylates, benzenesulfonates, phosphates and acetates.

The oxidation base(s) each advantageously represent(s) from 0.001% to 10% by weight relative to the total weight of the dyeing composition, and preferably from 0.005% to 5% by weight relative to the total weight of the dyeing composition.

The coupling agent(s), if they are present, each advantageously represent(s) from 0.001% to 10% by weight relative to the total weight of the dyeing composition, and preferably from 0.005% to 5% by weight relative to the total weight of the dyeing composition.

### Direct Dyes

The dyeing composition may optionally comprise one or more direct dyes.

Examples of suitable direct dyes that may be mentioned include azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (IIIa) and (III′a), the azo cationic dyes (IVa) and (IV′a) and the diazo cationic dyes (Va) below:

| | | |
|---|---|---|
| Het⁺ − C(R^{a}) = N − N(R^{b}) − Ar, | Het⁺ − N(R^{a}) − N = C(R^{b}) − Ar, | Het⁺ − N = N − Ar, An⁻ |
| An⁻ | An⁻ (III'a) | (IVa) |
| (IIIa) | | |
| Ar⁺ − N = N − Ar″, An⁻ | and | Het⁺ − N = N-Ar' − N = N − Ar, |
| (IV'a) | | An⁻ |
| | | (Va) |

in which formulae (IIIa), (III′a), (IVa), (IV′a) and (Va):
Het⁺ represents a cationic heteroaryl radical, preferably bearing an endocyclic cationic charge, such as imidazolium, indolium or pyridinium, which is optionally substituted, preferably with one or more (C₁-C₈)alkyl groups, such as methyl;
Ar⁺ represents an aryl radical, such as phenyl or naphthyl, bearing an exocyclic cationic charge, preferably ammonium, particularly tri(C₁-C₈)alkylammonium such as trimethylammonium;
Ar represents an aryl group, in particular phenyl, which is optionally substituted, preferably with one or more electron-donating groups, such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or, as a variant, Ar represents a julolidine group;
Ar′ represents an optionally substituted divalent (hetero)arylene group, such as phenylene, particularly para-phenylene, or naphthalene, which are optionally substituted, preferably with one or more (C₁-C₈)alkyl, hydroxyl or (C₁-C₈)alkoxy groups;
Ar″ represents an optionally substituted (hetero)aryl group, such as phenyl or pyrazolyl, which are optionally substituted, preferably with one or more (C₁-C₈)alkyl, hydroxyl, (di)(C₁-C₈)(alkyl)amino, (C₁-C₈)alkoxy or phenyl groups;
R^{a} and R^{b}, which may be identical or different, represent a hydrogen atom or a (C₁-C₈)alkyl group, which is optionally substituted, preferably with a hydroxyl group; or, as a variant, the substituent R^{a} with a substituent of Het⁺ and/or R^{b} with a substituent of Ar and/or R^{a} with R^{b} form, together with the atoms that bear them, a (hetero)cycloalkyl; in particular, R^{a} and R^{b} represent a hydrogen atom or a (C₁-C₄)alkyl group, which is optionally substituted with a hydroxyl group;
An⁻ represents an anionic counterion, such as mesylate or halide.

Mention may be made in particular of the azo and hydrazono cationic dyes bearing an endocyclic cationic charge of formulae (IIIa), (III′a) and (IVa) as defined previously. More particularly, those of formulae (IIIa), (III′a) and (IVa) derived from the dyes described in patent applications WO 95/15144, WO 95/01772 and EP-714954.

Preferably, the cationic part is derived from the following derivatives:

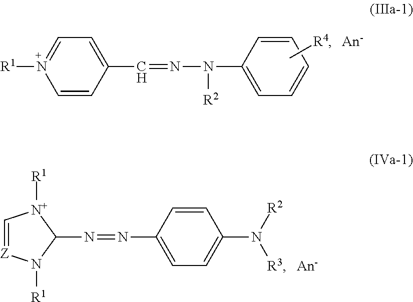

formulae (IIIa-1) and (IVa-1) with:
R¹ representing a (C₁-C₄)alkyl group such as methyl;
R² and R³, which may be identical or different, represent a hydrogen atom or a (C₁-C₄)alkyl group, such as methyl; and
R⁴ represents a hydrogen atom or an electron-donating group, such as an optionally substituted (C₁-C₃)alkyl, optionally substituted (C₁-C₃)alkoxy, or (di)(C₁-C₃)(alkyl)amino group optionally substituted on the alkyl group(s) with a hydroxyl group; in particular, R⁴ represents a hydrogen atom;
Z represents a CH group or a nitrogen atom, preferably CH;
An⁻ represents an anionic counterion, such as mesylate or halide.

In particular, the dye of formulae (IIIa-1) and (IVa-1) is chosen from Basic Red 51, Basic Yellow 87 and Basic Orange 31 or corresponding derivatives:

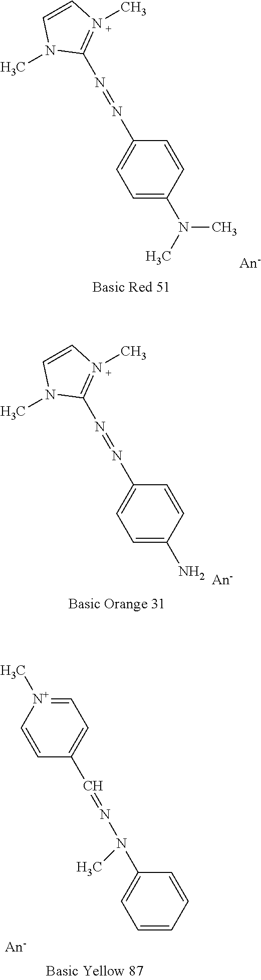

Among the natural direct dyes that may be used according to the invention, mention may be made of hennotannic acid, juglone, alizarin, purpurin, carminic acid, kermesic acid, purpurogallin, protocatechaldehyde, indigo, isatin, curcumin, spinulosin, apigenidin and orcein. Extracts or decoctions containing these natural dyes and in particular henna-based poultices or extracts may also be used.

When they are present, the direct dye(s) more particularly represent from 0.001% to 10% by weight and preferably from 0.005% to 5% by weight relative to the total weight of the dyeing composition.

The dyeing or bleaching composition may also optionally comprise one or more additives, different from the compounds described previously, among which mention may be made of cationic, anionic, non-ionic or amphoteric polymers or mixtures thereof, antidandruff agents, antiseborrhea agents, agents for preventing hair loss and/or for promoting hair regrowth, vitamins and provitamins including panthenol, sunscreens, mineral or organic pigments, sequestrants, plasticizers, solubilizers, acidifying agents, mineral or organic thickeners, notably polymeric thickeners, opacifiers or nacreous agents, antioxidants, hydroxy acids, fragrances, preserving agents, pigments and ceramides.

### Additional Characteristics

The amino acid(s) chosen from the compounds of formula (I₁) as defined above, salts thereof and mixtures thereof, or composition (A) as defined above can be applied to dry or wet keratin fibres.

### Bath Ratio

Composition (A) as defined above or the dyeing or bleaching composition may advantageously be applied to the keratin fibres in an amount ranging from 0.1 g to 10 g of amino acid(s) or of composition (A) or of dyeing or bleaching composition per gram of keratin fibres.

Preferably, composition (A) may be applied to the keratin fibres in an amount ranging from 0.2 g to 5 g of composition (A) per gram of keratin fibres.

### Leave-on Time

The amino acid(s) chosen from the compounds of formula (I₁) as defined above, salts thereof and mixtures thereof, or composition (A) as defined above can be left on the keratin fibres before carrying out the dyeing or bleaching process for a period ranging from 1 min to 60 min, preferably ranging from 3 min to 40 min, more preferentially ranging from 3 min to 20 min.

The leave-on time step may take place at a temperature ranging from 15° C. to 45° C., preferably at ambient temperature (25° C.). The leave-on time may take place under an occlusive system. A non-limiting example of an occlusive system that may be mentioned is an occlusive system of envelope type made of aluminium or plastic film or a hair cap with or without holes.

### EXAMPLES

The examples that follow allow the invention to be understood more clearly, without, however, being limiting in nature. In the examples that follow, unless otherwise indicated, all the amounts are shown as weight percentages relative to the total weight of the composition.

In the text that follows, the term alkaline solubility (AS) means the loss of mass of a sample of 100 mg of keratin fibres under the action of a decinormal sodium hydroxide solution for 30 minutes at 65° C.

### Compositions Used in the Examples

The following compositions were prepared:

### Pretreatment Compositions

TABLE 1

| Ingredients | A1 |
|---|---|
| Glycine | 10 |
| Denatured absolute ethyl alcohol | 10 |
| 50/50 dimethyldiallylammonium | 0.001 |
| chloride/acrylamide copolymer as an | (0.00009 AM) |
| aqueous solution | |
| (Merquat™ 550PR Polymer sold by the | |
| company Lubrizol) | |
| Oxyethylenated (40 OE) hydrogenated | 0.30 |
| castor oil | |
| Polydimethylsiloxane bearing aminoethyl | 0.50 |
| iminopropyl group as a stored non-ionic | (0.074 AM) |
| microemulsion | |
| (Belsil ADM LOG 1 sold by the company | |
| Wacker) | |
| Sodium hydroxide (100% pure sodium | qs PH = 9 |
| hydroxide reference concentration) | |
| Water | qs 100 |

AM: Active Material

TABLE 2

| Ingredients | A2 |
|---|---|
| Glycine | 10 |
| Denatured absolute ethyl alcohol | 10 |
| Sodium hydroxide (100% pure sodium | qs PH = 9 |
| hydroxide reference concentration) | |
| Water | qs 100 |

### Dyeing Bases

TABLE 3

| Ingredients | B1 |
|---|---|
| Oleic acid | 2.7 |
| Pure monoethanolamine | 0.63 |
| Cetylstearyl alcohol | 16.2 |
| Oleyl alcohol | 2.7 |
| Poly[(dimethyliminio)-1,3- | 5 |
| propanediyl(dimethyliminio)-1,6- | (3 AM) |
| hexanediyl dichloride] as an aqueous | |
| 60% solution | |
| Oxyethylenated oleocetyl alcohol (30 | 3.6 |
| OE) | |
| Ethylenediaminetetraacetic acid | 0.2 |
| Powdered sodium metabisulfite | 0.71 |
| N-Oleyldihydrosphingosine | 0.01 |
| 1-Methyl-2,5-diaminobenzene | 0.1 |
| 1-Hydroxy-4-aminobenzene | 0.038 |
| 1,3-Dihydroxybenzene (resorcinol) | 0.217 |
| 1-Hydroxy-3-aminobenzene | 0.03 |
| 1-ß-Hydroxyethyloxy-2,4- | 0.012 |
| diaminobenzene dihydrochloride | |
| Aqueous ammonia (20% ammonia | 13.4 |
| reference concentration) | |
| Fragrance | 0.5 |
| Water | qs 100 |

AM: Active Material

TABLE 4

| Ingredients | B2 |
|---|---|
| White mineral oil | 60 |
| Oxyethylenated (5 OE) decyl alcohol | 1.2 |
| Cetyl palmitate | 2 |
| Mixture of linear C18-24 fatty alcohols | 4.6 |
| (Nafol 2022 EN sold by the company | |
| Sasol) | |
| Oxyethylenated (20 OE) oleyl alcohol | 4 |
| Oxyethylenated (10 OE) oleyl alcohol | 1 |
| Oxyethylenated (60 OE) (C16/C18) | 0.01 |
| cetylstearyl alcohol myristyl glycol ether | |
| Carboxyvinyl polymer synthesized in an | 0.1 |
| ethyl acetate/cyclohexane mixture | |
| (Carbopol® 980 Polymer sold by the | |
| company Lubrizol) | |
| Glycerol | 5 |
| Vitamin C: Ascorbic acid | 0.12 |
| Ethylenediaminetetraacetic acid | 0.2 |
| Powdered sodium metabisulfite | 0.22 |
| Pure monoethanolamine | 4.26 |
| 1-Methyl-2,5-diaminobenzene | 0.2 |
| 1-Hydroxy-4-aminobenzene | 0.35 |
| 1,3-Dihydroxybenzene (resorcinol) | 0.08 |
| 2-Methyl-1,3-dihydroxybenzene (2- | 0.19 |
| methylresorcinol) | |
| 1-Hydroxy-3-aminobenzene | 0.1 |
| 2-Amino-3-hydroxypyridine | 0.12 |
| 1-Methyl-2-hydroxy-4-ß- | 0.043 |
| hydroxyethylaminobenzene | |
| 1-Methyl-2-hydroxy-4-aminobenzene | 0.043 |
| Water | qs 100 |

### Bleaching Powder

TABLE 5

| Ingredients | B3 |
|---|---|
| Potassium persulfate | 32.90 |
| Ammonium persulfate | 9.80 |
| Sodium silicate | 33.70 |
| Magnesium carbonate hydroxide | 9.20 |
| Disodium EDTA | 1.0 |
| Acrylates/C10-30 alkyl acrylate | 0.70 |
| crosspolymer | |
| Hydroxyethylcellulose | 0.70 |
| Glycine | 1.0 |
| Talc | 6.85 |
| Pigment | 0.25 |
| Dimethicone | 1.40 |
| Mineral oil | 2.50 |

### Oxidizing Compositions

TABLE 6

| Ingredients | D1 |
|---|---|
| Mixture of cetylstearyl | 2.85 |
| alcohol/oxyethylenated (30 OE) | |
| cetylstearyl alcohol | |
| (50% Linear 70/30 C13/C15) alkyl ether | 0.85 |
| carboxylic acid monoethanolamide (2 OE) | |
| Glycerol | 0.5 |
| Tetrasodium pyrophosphate. 10 H₂O | 0.04 |
| Sodium salicylate | 0.035 |
| Etidronic acid, tetrasodium salt, as a 30% | 0.2 |
| aqueous solution | |
| Hydrogen peroxide as a 50% solution (200 | 12 |
| vol. aqueous hydrogen peroxide solution) | |
| Water | qs 100 |

TABLE 7

| Ingredients | D2 |
|---|---|
| Cetylstearyl alcohol | 6 |
| Oxyethylenated (20 OE) stearyl alcohol | 5 |
| White mineral oil | 20 |
| Oxyethylenated (4 OE) rapeseed acid | 1.3 |
| amide | |
| Vitamin E: DL-α-Tocopherol | 0.1 |
| Glycerol | 0.5 |
| Poly[(dimethyliminio)-1,3- | 0.25 |
| propanediyl(dimethyliminio)-1,6- | |
| hexanediyl dichloride] as an aqueous | |
| 60% solution | |
| (Mexomer PO sold by the company | |
| Noveal) | |
| Tetrasodium pyrophosphate· 10 H₂O | 0.04 |
| Phosphoric acid | qs pH = 2.2 + 0.2 |
| Hydrogen peroxide as a 50% solution | 12 |
| (200 vol. aqueous hydrogen peroxide | |
| solution) | |
| Etidronic acid, tetrasodium salt, as a | 0.2 |
| 30% aqueous solution | |
| Sodium salicylate | 0.035 |
| Non-stabilized | |
| polydimethyldiallylammonium chloride at | 0.5 |
| 40% in water | |
| (Merquat™ 100 Polymer sold by the | |
| company Lubrizol) | |
| Water | qs 100 |

TABLE 8

| Ingredients | D3 |
|---|---|
| Tetrasodium pyrophosphate | 0.05 |
| Sodium salicylate | 0.05 |
| Tetrasodium etidronate | 0.12 |
| Mineral oil | 17.0 |
| Cetearyl alcohol | 3.15 |
| PEG-40 hydrogenated castor oil | 0.90 |
| Sodium cetearyl sulfate | 0.45 |
| Hydrogen peroxide | 9.0 |
| Phosphoric acid | qs pH = 4 + 0.2 |
| Water | qs 100 |

### Example 1

Three locks of moderately sensitized hair (AS 20) A, B and C were used in this example. Among these three locks, locks B and C were enriched with 500 ppm of copper using a copper(II) sulfate pentahydrate CuSO₄·5H₂O solution (minimum purity of 99%) from Prolabo®. This copper enrichment makes it possible to simulate the accumulation of copper on the hair that can occur over the course of successive washing operations due to the presence of high contents of copper in tap water.

Among these three locks:
lock A (not enriched with copper) underwent only a dyeing treatment according to the dyeing protocol described below;
lock B (enriched with copper) underwent only a dyeing treatment according to the dyeing protocol described below;
lock C (enriched with copper) underwent a pretreatment using composition A1 according to the pretreatment protocol described below followed by a dyeing treatment according to the dyeing protocol described below.

### Pretreatment Protocol

The lock is combed with a comb according to the following routine: 10 strokes with the coarse-toothed end, followed by 10 strokes with the fine-toothed end.

The lock is placed on a hotplate thermostatically regulated at 27° C.

The pretreatment composition A1 is applied to the lock. The bath ratio is 0.25 g of composition per 1 g of hair.

After a leave-on time of 5 minutes, the lock is blotted dry using a Kimtech 7505 absorbent paper towel.

### Dyeing Protocol

A dyeing composition is prepared by mixing 1 part by weight of composition B1 with 1.5 parts by weight of composition D1 and is then applied to the lock. The bath ratio is 3 g of composition per 1 g of hair.

The lock is placed on a hotplate thermostatically regulated at 27° C.

After a leave-on time of 35 minutes, the lock is rinsed and washed with DOP shampoo.

The lock is dried in an oven regulated at 60° C. for 30 minutes.

On exiting the oven, the lock is combed: 10 strokes with the coarse-toothed end and 10 strokes with the fine-toothed end.

### Measurements

The colouring of the hair was evaluated in the L*a*b* system, using a Konica Minolta CM 2600d spectrocolorimeter (illuminant D65, angle 10°, specular component included).

In this L*a*b* system, L* represents the intensity of the colour, a* indicates the green/red colour axis and b* the blue/yellow colour axis.

The difference in colour build-up compared to lock A not enriched with copper was calculated according to the following equation:

ΔE=\sqrt{(L*₁−L*₀)²+(a*₁−a*₀)²+(b*₁−b*₀)²}

with:
L*₁, a*₁, b*₁ representing the colorimetric values of lock B or C
L*₀, a*₀, b*₀ representing the colorimetric values of lock A.

Furthermore, a visual evaluation of the uniformity of the colour was performed.

### Results

### Difference in Colour Build-Up Compared to Lock A

TABLE 9

| | Copper | | | | | |
|---|---|---|---|---|---|---|
| | doping | | | | | |
| Type of lock | 500 ppm | Pretreatment | L* | a* | b* | ΔE |
| Lock A | No | No | 37.73 | 5.09 | 15.05 | / |
| (Expected result) | | | | | | |
| Lock B | Yes | No | 44.43 | 4.45 | 17.33 | 7.11 |
| Lock C | Yes | Yes | 40.09 | 4.23 | 15.08 | 2.51 |
| (Invention) | | | | | | |

The results show that the pretreatment with composition A1 makes it possible to improve the colouring of lock C enriched with copper, with a colour build-up and intensity that are close to those observed for lock A not enriched with copper, which is not the case for lock B. Composition A1 thus makes it possible to reduce the colour differences between a copper-free lock and a lock polluted with copper. Better colour uniformity will thus be observed on a head of hair polluted with copper that can, depending on areas, exhibit different amounts of copper.

### Visual Evaluation of the Locks

TABLE 10

| | Copper | | |
|---|---|---|---|
| Type of | doping | | |
| lock | 500 ppm | Pretreatment | Visual evaluation of the colour |
| Lock A | No | No | Colour uniform |
| Lock B | Yes | No | Colour non-uniform with light |
| | | | blonde ″spots″ and darker ″spots″ |
| Lock C | Yes | Yes | Colour uniform |

Better colour uniformity is observed, i.e. a uniform colour all along the lock without “spots” of different colours for lock C enriched with copper and pretreated with composition A1, as for lock A, this not being the case for lock B.

### Example 2

Three locks of moderately sensitized hair (SA20) D, E and F were used in this example. Among these three locks, locks E and F were enriched with 200 ppm of copper using a copper(II) sulfate pentahydrate CuSO₄·5H₂O solution (minimum purity of 99%) from Prolabo®. This copper enrichment makes it possible to simulate the accumulation of copper on the hair that can occur over the course of successive washing operations due to the presence of high contents of copper in tap water.

Among these three locks:
lock D (not enriched with copper) underwent only a dyeing treatment according to the dyeing protocol described below;
lock E (enriched with copper) underwent only a dyeing treatment according to the dyeing protocol described below;
lock F (enriched with copper) underwent a pretreatment using composition A1 according to the pretreatment protocol described below followed by a dyeing treatment according to the dyeing protocol described below.

### Pretreatment Protocol

The lock is combed with a comb according to the following routine: 10 strokes with the coarse-toothed end, followed by 10 strokes with the fine-toothed end.

The lock is placed on a hotplate thermostatically regulated at 27° C.

The pretreatment composition A1 is applied to the lock. The bath ratio is 0.25 g of composition per 1 g of hair.

After a leave-on time of 5 minutes, the lock is blotted dry using a Kimtech 7505 absorbent paper towel.

### Dyeing Protocol

A dyeing composition is prepared by mixing 1 part by weight of composition B2 with 1 part by weight of composition D2 and is then applied to the lock. The bath ratio is 3 g of composition per 1 g of hair.

The lock is placed on a hotplate thermostatically regulated at 27° C.

After a leave-on time of 35 minutes, the lock is rinsed and washed with DOP shampoo.

The lock is dried in an oven regulated at 60° C. for 30 minutes.

On exiting the oven, the lock is combed: 10 strokes with the coarse-toothed end and 10 strokes with the fine-toothed end.

### Measurements

The colouring of the hair was evaluated in the L*a*b* system, using a Konica Minolta CM 2600d spectrocolorimeter (illuminant D65, angle 10°, specular component included).

In this L*a*b* system, L* represents the intensity of the colour, a* indicates the green/red colour axis and b* the blue/yellow colour axis.

The difference in colour build-up compared to lock D not enriched with copper was calculated according to the following equation:

ΔE=\sqrt{(L*₁−L*₀)²+(a*₁−a*₀)²+(b*₁−b*₀)²}

with:
L*₁, a*₁, b*₁ representing the colorimetric values of lock E or F
L*₀, a*₀, b*₀ representing the colorimetric values of lock D.

### Results

TABLE 11

| | Copper | | | | | |
|---|---|---|---|---|---|---|
| | doping | | | | | |
| Type of lock | 200 ppm | Pretreatment | L* | a* | b* | ΔE |
| Lock D | No | No | 24.39 | 12.01 | 13.17 | / |
| (Expected result) | | | | | | |
| Lock E | Yes | No | 31.25 | 10.98 | 16.5 | 7.69 |
| Lock F | Yes | Yes | 25.01 | 10.44 | 11.85 | 2.14 |
| (Invention) | | | | | | |

The results show that the pretreatment with composition A1 makes it possible to improve the colouring of lock F enriched with copper, with a colour build-up and intensity that are close to those observed for lock D not enriched with copper, which is not the case for lock E. Composition A1 thus makes it possible to reduce the colour differences between a copper-free lock and a lock polluted with copper. Better colour uniformity will thus be observed on a head of hair polluted with copper that can, depending on areas, exhibit different amounts of copper.

### Example 3

Two locks of permanent-waved natural Caucasian hair containing 90% white hairs (WP90) not enriched with copper were used in this example.

Among these two locks:
lock G underwent only a dyeing treatment according to the dyeing protocol described below;
lock H underwent a pretreatment using composition A1 according to the pretreatment protocol described below followed by a dyeing treatment according to the dyeing protocol described below.

### Pretreatment Protocol

The lock is combed with a comb according to the following routine: 10 strokes with the coarse-toothed end, followed by 10 strokes with the fine-toothed end.

The lock is placed on a hotplate thermostatically regulated at 27° C.

The pretreatment composition A1 is applied to the lock. The bath ratio is 0.25 g of composition per 1 g of hair.

After a leave-on time of 5 minutes, the lock is blotted dry using a Kimtech 7505 absorbent paper towel.

### Dyeing Protocol

A dyeing composition is prepared by mixing 1 part by weight of composition B2 with 1 part by weight of composition D2 and is then applied to the lock. The bath ratio is 3 g of composition per 1 g of hair.

The lock is placed on a hotplate thermostatically regulated at 27° C.

After a leave-on time of 35 minutes, the lock is rinsed and washed with DOP shampoo.

The lock is dried in an oven regulated at 60° C. for 30 minutes.

On exiting the oven, the lock is combed: 10 strokes with the coarse-toothed end and 10 strokes with the fine-toothed end.

### Measurements

The colouring of the hair was evaluated in the L*a*b* system, using a Konica Minolta CM 2600d spectrocolorimeter (illuminant D65, angle 10°, specular component included).

In this L*a*b* system, L* represents the intensity of the colour, a* indicates the green/red colour axis and b* the blue/yellow colour axis.

The colour build-up between lock G or H and an undyed WP90 lock (control lock) was calculated according to the following equation:

dE*=\sqrt{(L*₁−L*₀)²+(a*₁−a*₀)²+(b*₁−b*₀)²}

with:
L*₁, a*₁, b*₁ representing the colorimetric values of lock G or H
L*₀, a*₀, b*₀ representing the colorimetric values of the undyed WP90 lock.

### Results

TABLE 12

| Type of lock | L* | a* | b* | dE* |
|---|---|---|---|---|
| Undyed WP90 lock | 60.90 | 0.88 | 13.86 | / |
| Lock G | 28.49 | 14.05 | 15.60 | 35.03 |
| Lock H | 25.63 | 13.88 | 13.75 | 37.59 |
| (Invention) | | | | |

The results show that the pretreatment with composition A1 makes it possible to obtain a better colour build-up and a better colour intensity on permanent-waved hair and therefore better dyeing performance levels.

### Example 4

Two locks of natural Caucasian hair, with a brown tone depth (HT4), not enriched with copper were used in this example.

Among these two locks:
lock I underwent only a bleaching treatment according to the bleaching protocol described below;
lock J underwent a pretreatment using composition A2 according to the pretreatment protocol described below followed by a bleaching treatment according to the bleaching protocol described below.

### Pretreatment Protocol

The lock is placed on a hotplate thermostatically regulated at 33° C.

The pretreatment composition A2 is applied to the lock. The bath ratio is 2 g of composition per 1 g of hair.

After a leave-on time of 5 minutes, the lock is blotted dry using a Kimtech 7505 absorbent paper towel.

### Bleaching Protocol

A bleaching composition is prepared by mixing 1 part by weight of composition B3 with 1.5 parts by weight of composition D3 and was then applied to the lock. The bath ratio is 10 g of composition per 1 g of hair.

The lock is then wrapped in aluminium foil and then returned onto a hotplate thermostatically regulated at 33° C.

After a leave-on time of 50 min, the lock is rinsed and washed with L'Oréal Blond Studio shampoo.

The lock is dried in an oven regulated at 60° C. for 20 min.

### Measurements

The colouring of the hair was evaluated in the L*a*b* system, using a Konica Minolta CM 2600d spectrocolorimeter (illuminant D65, angle 10°, specular component included).

In this L*a*b* system, L* represents the intensity of the colour, a* indicates the green/red colour axis and b* the blue/yellow colour axis.

The colour build-up between lock I or J and a non-bleached HT4 lock (control lock) was calculated according to the following equation:

dE*=\sqrt{(L*₁−L*₀)²+(a*₁−a*₀)²+(b*₁−b*₀)²}

with:
L*₁, a*₁, b*₁ representing the colorimetric values of lock I or J
L*₀, a*₀, b*₀ representing the colorimetric values of the non-bleached HT4 lock.

TABLE 13

| Type of lock | L' | a* | b* | dE* |
|---|---|---|---|---|
| Non-bleached HT4 lock | 8.7 | 3.7 | 8.2 | / |
| Lock I | 59.28 | 11.25 | 31.2 | 56.07 |
| Lock J | 62.34 | 9.5 | 30.92 | 58.65 |
| (Invention) | | | | |

The results show that the pretreatment with composition A2 makes it possible to obtain better bleaching of the fibre and a less coppery glint.

1-20. (canceled)

21. A method for treating keratin fibers comprising applying to the keratin fibers, a composition (A) comprising at least one amino acid chosen from compounds of formula (I₁), salts thereof, or mixtures thereof: 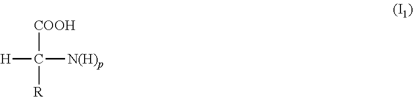
wherein in formula (I₁):
p is an integer equal to 1 or 2;
when p=1, R forms with the nitrogen atom a saturated 5- to 8-membered heterocycle, optionally substituted with at least one group chosen from i) a hydroxyl group, ii) a (C₁-C₄)alkyl group, or iii) combinations of two or more thereof;
when p=2, R represents:
a hydrogen atom; or
a (C₁-C₁₂)alkyl group i) interrupted with at least one heteroatom or group chosen from —S—, —NH—, or —C(NH)— ii) substituted with at least one group chosen from a hydroxyl group, an amino group, or an —NH—C(NH)—NH₂ group, or (iii) combinations or two or more thereof,
wherein composition (A) is applied a pretreatment for a process for dyeing or bleaching keratin fibers.

22. The method of claim 21, wherein the at least one amino acid is chosen from glycine, proline, methionine, serine, arginine, lysine, salts thereof, or mixtures of two or more thereof.

23. The method of claim 21, wherein the total amount of amino acid(s) is at least 5% by weight, relative to the total weight of composition (A).

24. The method of claim 21, wherein composition (A) further comprises at least one monoalcohol.

25. The method of claim 24, wherein the total amount of monoalcohol(s) is at least 5% by weight, relative to the total weight of composition (A).

26. The method of claim 21, wherein the pH of composition (A) ranges from 2 to 11.

27. The method of claim 21, wherein composition (A) further comprises at least one cationic polymer.

28. The method of claim 27, wherein the at least one cationic polymer is chosen from homopolymers or copolymers comprising one or more units of chosen from formula (I) or (II): 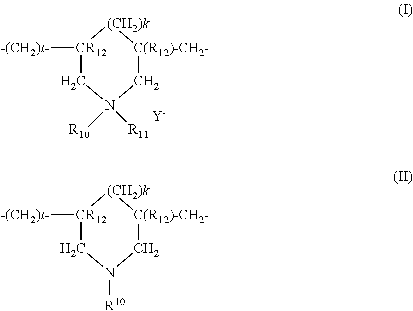
wherein:
k and t are equal to 0 or 1, and the sum of k+t is 1;
R₁₂ represents a hydrogen atom or a methyl group;
R₁₀ and R₁₁ are independently chosen from a (C₁-C₆)alkyl group, a C₁-C₅ hydroxyalkyl group, a C₁-C₄ amidoalkyl group; or R₁₀ and R₁₁ represent, together with the nitrogen atom to which they are attached, a heterocyclic group;
Y⁻ is an anion.

29. The method of claim 27, wherein the total amount of cationic polymer(s) ranges from 0.00001% to 5% by weight, relative to the total weight of composition (A).

30. The method of claim 21, wherein composition (A) further comprises at least one silicone.

31. The method of claim 30, wherein the at least one silicone is chosen from amino silicones of formula (F): 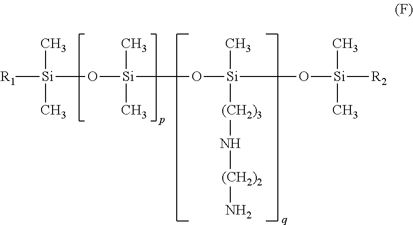
wherein:
p and q are integers such that the sum of (p+q) ranges from 1 to 1000;
R₁ and R₂ are independently chosen from a hydroxyl or C₁-C₄ alkoxy radical, wherein at least one of the radicals R₁ or R₂ represents an alkoxy radical.

32. The method of claim 30, wherein the total amount of silicone(s) ranges from 0.001% to 10% by weight, relative to the total weight of composition (A).

33. The method of claim 21, wherein composition (A) further comprises at least one coloring agent, at least one reducing agent, or combinations of two or more thereof present in a total amount of less than 0.1% by weight, relative to the total weight of composition (A).

34. The method of claim 21, wherein composition (A) further comprises at least one anionic surfactant in a total amount of less than 0.1% by weight, relative to the total weight of composition (A).

35. The method of claim 21, further comprising applying a dyeing or bleaching composition to the keratin fibers.

36. The method of claim 35, wherein the dyeing or bleaching composition comprises at least one chemical oxidizing agent.

37. The method of claim 35, wherein the dyeing composition comprises at least one coloring agent chosen from oxidation dyes, direct dyes, or mixtures thereof.

38. The method of claim 21, wherein the keratin fibers are sensitized keratin fibers.

39. The method of claim 21, wherein the application of composition (A) improves:
(i) the dyeing or bleaching of the keratin fibers,
(ii) the color build-up of the keratin fibers,
(iii) the color intensity of the keratin fibers,
(iv) the color uniformity of the keratin fibers,
(v) the lightening of the keratin fibers,
or combinations of two or more thereof.

40. A composition for treating keratin fibers, comprising:
5% to 15% of at least one amino acid;
5% to 20% of at least one monoalcohol;
at least one cationic polymer; and
at least one silicone,
wherein the pH of the composition ranges from 2 to 11.
